(19) United States
(12) Patent Application Publication
(10) Pub. No.: US 2025/0041230 A1
(43) Pub. Date: Feb. 6, 2025

(54) NEW COATING SYSTEM (II)

(71) Applicant: DSM IP Assets B.V., Heerlen (NL)

(72) Inventors: Jimmy DOUCOURE, Kaiseraugst (CH); Elger FUNDA, Kaiseraugst (CH); Olivia Brigitte VIDONI, Kaiseraugst (CH); Alexandru ZABARA, Kaiseraugst (CH)

(21) Appl. No.: 18/719,753

(22) PCT Filed: Dec. 15, 2022
(86) PCT No.: PCT/EP2022/086012

§ 371 Date: Jun. 13, 2024

(30) Foreign Application Priority Data
Dec. 17, 2021 (EP) 21215554.3

(51) Int. Cl. A61K 9/50

(52) U.S. Cl. CPC A61K 9/5015; A61K 9/501; A61K 9/5026; A61K 9/5047; A61K 9/5057; A61K 9/5063; A61K 9/5089

(57) ABSTRACT

The present invention relates to a new coating system useful for coating particles, which comprise an active ingredient. Such coated particles, when consumed, do show a controlled release in the human or animal body.

The present invention relates to a new coating system useful for coating particles, which comprise an active ingredient.

Such coated particles, when consumed, do show a controlled release in the human or animal body.

Controlled release is a very important property for particles (such as i.e., tablet, granules etc), in that the active ingredient, which is in the particle, can be delivered to the right place in the human or animal gastrointestinal tract, where the active ingredient is then released.

Such active ingredients can be any commonly used ingredients, such as i.e., a pharmaceutical compound (a drug), a vitamin, a mineral, probiotic, enzyme, eubiotic, plant extract and nutraceutical.

The goal was to find a coating layer that allows the release of the active ingredient inside the particle after passing the stomach in the small intestine.

Surprisingly, it was found that when using a coating system to coat a particle, wherein the coating system is an emulsion consisting of an
(i) aqueous solution comprising
   a. water, and
   b. at least one emulsifier, and
   c. at least one plasticizer, and
   d. at least one film forming agent, and
   e. at least one pH adjuster, and
(ii) oily phase comprising
   a. at least one enteric coating material, and
   b. at least one polyunsaturated fatty acid, and
   c. optionally at least one unsaturated fatty acid, then the so coated particle, shows a delayed release so that the release takes place where it is desired.

Therefore, the present invention relates to a coating system (CS) consisting of an
(i) aqueous solution comprising
   a. water, and
   b. at least one emulsifier, and
   c. at least one plasticizer, and
   d. at least one film forming agent, and
   e. at least one pH adjuster, and
(ii) oily phase comprising
   a. at least one enteric coating material, and
   b. at least one polyunsaturated fatty acid, and
   c. optionally at least one saturated fatty acid.

Therefore, the present invention relates to a coating system (CS1), which is the coating system (CS), wherein the at least one emulsifier is gelatin.

The gelatin can be from any source, such as porcine (pork), bovine (beef or cattle), and fish.

Therefore, the present invention relates to a coating system (CS2), which is the coating system (CS) or (CS1), wherein the at least one plasticizer is chosen from the group consisting of diesters of acids, triesters of acids, diesters of alcohols, triesters of alcohols, polyols and polyethylene glycols.

Therefore, the present invention relates to a coating system (CS2′), which is the coating system (CS) or (CS1), wherein the at least one plasticizer is chosen from the group consisting of triethyl citrate, tributyl citrate, acetyl triethyl citrate and glycerol.

Therefore, the present invention relates to a coating system (CS3), which is the coating system (CS), (CS1), (CS2) or (CS2′), wherein the at least one film forming agent is shellac and/or shellac ammonium salt, polyvinylpyrrolidone (PVP), acrylates, acrylamides, and copolymers thereof.

Therefore, the present invention relates to a coating system (CS4), which is the coating system (CS), (CS1), (CS2), (CS2′) or (CS3), wherein the at least one pH adjuster is chosen from the group consisting of NaOH and KOH.

Therefore, the present invention relates to a coating system (CS5), which is the coating system (CS), (CS1), (CS2), (CS2′), (CS3) or (CS4), wherein the at least one enteric coating is a wax.

Therefore, the present invention relates to a coating system (CS5′), which is the coating system (CS), (CS1), (CS2), (CS2′), (CS3) or (CS4), wherein the enteric coating is Candelilla wax.

Candelilla wax is a wax derived from the leaves of the small Candelilla shrub native to northern Mexico and the southwestern United States, Euphorbia Cerifera and Euphorbia antisyphilitica, from the family Euphorbiaceae. It is yellowish-brown, hard, brittle, aromatic, and opaque to translucent. Candelilla wax consists of mainly hydrocarbons (about 50%, chains with 29-33 carbons), esters of higher molecular weight (20-29%), free acids (7-9%), and resins (12-14%, mainly triterpenoid esters) Therefore, the present invention relates to a coating system (CS6), which is the coating system (CS), (CS1), (CS2), (CS2′), (CS3), (CS4), (CS5) or (CS5′), wherein the at least one polyunsaturated fatty acid has 16 to 24 carbon atoms.

Therefore, the present invention relates to a coating system (CS6′), which is the coating system (CS), (CS1), (CS2), (CS2′), (CS3), (CS4), (CS5) or (CS5′), wherein the at least one polyunsaturated fatty acid is chosen from the group consisting of omega-3 fatty acids, omega-6 fatty acids, omega-9 fatty acids and conjugated fatty acids.

Therefore, the present invention relates to a coating system (CS6″), which is the coating system (CS), (CS1), (CS2), (CS2′), (CS3), (CS4), (CS5) or (CS5′), wherein the at least one polyunsaturated fatty acid is chosen from the group consisting of oleic acid and linoleic acid.

Therefore, the present invention relates to a coating system (CS7), which is the coating system (CS), (CS1), (CS2), (CS2′), (CS3), (CS4), (CS5), (CS5′), (CS6), (CS6′) or (CS6″), wherein the at least one saturated fatty acid has 16 to 24 carbon atoms.

Therefore, the present invention relates to a coating system (CS7′), which is the coating system (CS), (CS1), (CS2), (CS2′), (CS3), (CS4), (CS5), (CS5′), (CS6), (CS6′) or (CS6″) wherein the at least one saturated fatty acid is chosen from the group consisting of lauric acid, myristic acid, palmitic acid and stearic acid.

Usually, the emulsion consists of 50-80 weight-% (wt-%), based on the total weight of the emulsion, of the aqueous (water) phase and 20-50 wt-%, based on the total weight of the emulsion, of the oily phase.

Therefore, the present invention relates to a coating system (CS8), which is the coating system (CS), (CS1), (CS2), (CS2′), (CS3), (CS4), (CS5), (CS5′), (CS6), (CS6′), (CS6″), (CS7) or (CS7′), wherein the emulsion consists of 50-80 weight-% (wt-%), based on the total weight of the emulsion, of the aqueous (water) phase and 20-50 wt-%, based on the total weight of the emulsion, of the oily phase.

The aqueous phase of the emulsion comprises
65-90 wt-%, based on the total weight of the aqueous phase, of water and
0.5-10 wt-%, based on the total weight of the aqueous phase, of at least one emulsifier, and
0.5-10 wt-%, based on the total weight of the aqueous phase, of at least one plasticizer, and
2-15 wt-%, based on the total weight of the aqueous phase, of at least one film forming agent, and
2-15 wt-%, based on the total weight of the aqueous phase, of at least one pH adjuster, and
up to 15 wt-%, based on the total weight of the aqueous phase, of at least one further auxiliary agent.

Such auxiliary agents can be i.e. colors, antifoaming agents, flavors, and antioxidants etc.

The aqueous phase of the emulsion consists of
65-90 wt-%, based on the total weight of the aqueous phase, of water, and
0.5-10 wt-%, based on the total weight of the aqueous phase, of at least one emulsifier, and
0.5-10 wt-%, based on the total weight of the aqueous phase, of at least one plasticizer, and
2-15 wt-%, based on the total weight of the aqueous phase, of at least one film forming agent, and
2-15 wt-%, based on the total weight of the aqueous phase, of at least one pH adjuster, and
up to 15 wt-%, based on the total weight of the aqueous phase, of at least one further auxiliary agent.

The aqueous phase of the emulsion consists of
65-90 wt-%, based on the total weight of the aqueous phase, of water, and
0.5-10 wt-%, based on the total weight of the aqueous phase, of at least one emulsifier, and
0.5-10 wt-%, based on the total weight of the aqueous phase, of at least one plasticizer, and
2-15 wt-%, based on the total weight of the aqueous phase, of at least one film forming agent, and
2-15 wt-%, based on the total weight of the aqueous phase, of at least one pH adjuster.

Preferably, the aqueous phase of the emulsion comprises
70-90 wt-%, based on the total weight of the aqueous phase, of water, and
0.5-5 wt-%, based on the total weight of the aqueous phase, of at least one emulsifier, and
0.5-5 wt-%, based on the total weight of the aqueous phase, of at least one plasticizer, and
2-12 wt-%, based on the total weight of the aqueous phase, of at least one film forming agent, and
2-12 wt-%, based on the total weight of the aqueous phase, of at least one pH adjuster, and
up to 15 wt-%, based on the total weight of the aqueous phase, of at least one further auxiliary agent.

Preferably, the aqueous phase of the emulsion consists of
70-90 wt-%, based on the total weight of the aqueous phase, of water, and
0.5-5 wt-%, based on the total weight of the aqueous phase, of at least one emulsifier, and
0.5-5 wt-%, based on the total weight of the aqueous phase, of at least one plasticizer, and
2-12 wt-%, based on the total weight of the aqueous phase, of at least one film forming agent, and
2-12 wt-%, based on the total weight of the aqueous phase, of at least one pH adjuster, and
up to 15 wt-%, based on the total weight of the aqueous phase, of at least one further auxiliary agent.

Preferably, the aqueous phase of the emulsion consists of
70-90 wt-%, based on the total weight of the aqueous phase, of water, and
0.5-5 wt-%, based on the total weight of the aqueous phase, of at least one emulsifier, and
0.5-5 wt-%, based on the total weight of the aqueous phase, of at least one plasticizer, and
2-12 wt-%, based on the total weight of the aqueous phase, of at least one film forming agent, and
2-12 wt-%, based on the total weight of the aqueous phase, of at least one pH adjuster.

Therefore, the present invention relates to a coating system (CS9), which is the coating system (CS), (CS1), (CS2), (CS2′), (CS3), (CS4), (CS5), (CS5′), (CS6), (CS6′), (CS6″), (CS7), (CS7′) or (CS8), wherein the aqueous phase of the emulsion comprises
65-90 wt-%, based on the total weight of the aqueous phase, of water, and
0.5-10 wt-%, based on the total weight of the aqueous phase, of at least one emulsifier, and
0.5-10 wt-%, based on the total weight of the aqueous phase, of at least one plasticizer, such as glycerol, and
2-15 wt-%, based on the total weight of the aqueous phase, of at least one film forming agent, and
2-15 wt-%, based on the total weight of the aqueous phase, of at least one pH adjuster, and
up to 15 wt-%, based on the total weight of the aqueous phase, of at least one further auxiliary agent.

Therefore, the present invention relates to a coating system (CS9′), which is the coating system (CS), (CS1), (CS2), (CS2′), (CS3), (CS4), (CS5), (CS5′), (CS6), (CS6′), (CS6″), (CS7), (CS7′) or (CS8), wherein the aqueous phase of the emulsion consists of
65-90 wt-%, based on the total weight of the aqueous phase, of water, and
0.5-10 wt-%, based on the total weight of the aqueous phase, of at least one emulsifier, and
0.5-10 wt-%, based on the total weight of the aqueous phase, of at least one plasticizer, and
2-15 wt-%, based on the total weight of the aqueous phase, of at least one film forming agent, and
2-15 wt-%, based on the total weight of the aqueous phase, of at least one pH adjuster, and
up to 15 wt-%, based on the total weight of the aqueous phase, of at least one further auxiliary agent.

Therefore, the present invention relates to a coating system (CS9″), which is the coating system (CS), (CS1), (CS2), (CS2′), (CS3), (CS4), (CS5), (CS5′), (CS6), (CS6′), (CS6″), (CS7), (CS7′) or (CS8), wherein the aqueous phase of the emulsion consists of
65-90 wt-%, based on the total weight of the aqueous phase, of water, and
0.5-10 wt-%, based on the total weight of the aqueous phase, of at least one emulsifier, and
0.5-10 wt-%, based on the total weight of the aqueous phase, of at least one plasticizer, and
2-15 wt-%, based on the total weight of the aqueous phase, of at least one film forming agent, and
2-15 wt-%, based on the total weight of the aqueous phase, of at least one pH adjuster.

Therefore, the present invention relates to a coating system (CS9′″), which is the coating system (CS), (CS1), (CS2), (CS2′), (CS3), (CS4), (CS5), (CS5′), (CS6), (CS6′), (CS6″), (CS7), (CS7′) or (CS8), wherein the aqueous phase of the emulsion comprises
70-90 wt-%, based on the total weight of the aqueous phase, of water, and
0.5-5 wt-%, based on the total weight of the aqueous phase, of at least one emulsifier, and
0.5-5 wt-%, based on the total weight of the aqueous phase, of at least one plasticizer, and
2-12 wt-%, based on the total weight of the aqueous phase, of at least one film forming agent, and
2-12 wt-%, based on the total weight of the aqueous phase, of at least one pH adjuster, and
up to 15 wt-%, based on the total weight of the aqueous phase, of at least one further auxiliary agent.

Therefore, the present invention relates to a coating system (CS9″ ″), which is the coating system (CS), (CS1), (CS2), (CS2′), (CS3), (CS4), (CS5), (CS5′), (CS6), (CS6′), (CS6″), (CS7), (CS7′) or (CS8), wherein the aqueous phase of the emulsion consists of
70-90 wt-%, based on the total weight of the aqueous phase, of water, and
0.5-5 wt-%, based on the total weight of the aqueous phase, of at least one emulsifier, and
0.5-5 wt-%, based on the total weight of the aqueous phase, of at least one plasticizer, and
2-12 wt-%, based on the total weight of the aqueous phase, of at least one film forming agent, and
2-12 wt-%, based on the total weight of the aqueous phase, of at least one pH adjuster, and
up to 15 wt-%, based on the total weight of the aqueous phase, of at least one further auxiliary agent.

Therefore, the present invention relates to a coating system (CS9″″′), which is the coating system (CS), (CS1), (CS2), (CS2′), (CS3), (CS4), (CS5), (CS5′), (CS6), (CS6′), (CS6″), (CS7), (CS7′) or (CS8), wherein the aqueous phase of the emulsion consists of
70-90 wt-%, based on the total weight of the aqueous phase, of water and
0.5-5 wt-%, based on the total weight of the aqueous phase, of at least one emulsifier, and
0.5-5 wt-%, based on the total weight of the aqueous phase, of at least one plasticizer, and
2-12 wt-%, based on the total weight of the aqueous phase, of at least one film forming agent, and
2-12 wt-%, based on the total weight of the aqueous phase, of at least one pH adjuster.

The oily phase of the emulsion comprises
60-95 wt-%, based on the total weight of the oily phase, of at least one enteric coating material, and
2-20 wt-%, based on the total weight of the oily phase, of at least one polyunsaturated fatty acid, and
optionally 1-20 wt-%, based on the total weight of the oily phase, of at least one saturated fatty acid, and
up to 15 wt-%, based on the total weight of the oily phase, of at least one further auxiliary agent.

The oily phase of the emulsion consists of
60-95 wt-%, based on the total weight of the oily phase, of at least one enteric coating material, and
2-20 wt-%, based on the total weight of the oily phase, of at least one polyunsaturated fatty acid, and
optionally 1-20 wt-%, based on the total weight of the oily phase, of at least one saturated fatty acid, and
up to 15 wt-%, based on the total weight of the oily phase, of at least one further auxiliary agent.

The oily phase of the emulsion consists of
50-95 wt-%, based on the total weight of the oily phase, of at least one enteric coating material, and
2-25 wt-%, based on the total weight of the oily phase, of at least one polyunsaturated fatty acid, and
optionally 1-25 wt-%, based on the total weight of the oily phase, of at least one saturated fatty acid, and
up to 15 wt-%, based on the total weight of the oily phase, of at least one further auxiliary agent.

Preferably, the oily phase of the emulsion comprises
60-95 wt-%, based on the total weight of the oily phase, of at least one enteric coating material, and
2-20 wt-%, based on the total weight of the oily phase, of at least one polyunsaturated fatty acid, and
optionally 1-20 wt-%, based on the total weight of the oily phase, of at least one saturated fatty acid, and
up to 15 wt-%, based on the total weight of the oily phase, of at least one further auxiliary agent.

Preferably, the oily phase of the emulsion comprises
60-95 wt-%, based on the total weight of the oily phase, of at least one enteric coating material, and
2-20 wt-%, based on the total weight of the oily phase, of at least one polyunsaturated fatty acid, and
optionally 1-20 wt-%, based on the total weight of the oily phase, of at least one saturated fatty acid.

Preferably, the oily phase of the emulsion consists of
60-95 wt-%, based on the total weight of the oily phase, of at least one enteric coating material, and
2-20 wt-%, based on the total weight of the oily phase, of at least one polyunsaturated fatty acid, and
optionally 1-20 wt-%, based on the total weight of the oily phase, of at least one saturated fatty acid, and
up to 15 wt-%, based on the total weight of the oily phase, of at least one further auxiliary agent.

Preferably, the oily phase of the emulsion consists of
60-95 wt-%, based on the total weight of the oily phase, of at least one enteric coating material, and
2-20 wt-%, based on the total weight of the oily phase, of at least one polyunsaturated fatty acid, and
optionally 1-20 wt-%, based on the total weight of the oily phase, of at least one saturated fatty acid.

Therefore, the present invention relates to a coating system (CS10), which is the coating system (CS), (CS1), (CS2), (CS2′), (CS3), (CS4), (CS5), (CS5′), (CS6), (CS6′), (CS6″), (CS7), (CS7′), (CS8), (CS9), (CS9′), (CS9″), (CS9′″), (CS9″ ″) or (CS9″″′), wherein the oily phase of the emulsion comprises
60-95 wt-%, based on the total weight of the oily phase, of at least one enteric coating material, and
2-20 wt-%, based on the total weight of the oily phase, of at least one polyunsaturated fatty acid, and
optionally 1-20 wt-%, based on the total weight of the oily phase, of at least one saturated fatty acid, and
up to 15 wt-%, based on the total weight of the oily phase, of at least one further auxiliary agent.

Therefore, the present invention relates to a coating system (CS10′), which is the coating system (CS), (CS1), (CS2), (CS2′), (CS3), (CS4), (CS5), (CS5′), (CS6), (CS6′), (CS6″), (CS7), (CS7′), (CS8), (CS9), (CS9′), (CS9″), (CS9′″), (CS9″ ″) or (CS9″″′), wherein the oily phase of the emulsion consists of
60-95 wt-%, based on the total weight of the oily phase, of at least one enteric coating material, and
2-20 wt-%, based on the total weight of the oily phase, of at least one polyunsaturated fatty acid, and
optionally 1-20 wt-%, based on the total weight of the oily phase, of at least one saturated fatty acid, and
up to 15 wt-%, based on the total weight of the oily phase, of at least one further auxiliary agent.

Therefore, the present invention relates to a coating system (CS10″), which is the coating system (CS), (CS1), (CS2), (CS2′), (CS3), (CS4), (CS5), (CS5′), (CS6), (CS6′), (CS6″), (CS7), (CS7′), (CS8), (CS9), (CS9′), (CS9″), (CS9′″), (CS9″ ″) or (CS9″″′), wherein the oily phase of the emulsion consists of
50-95 wt-%, based on the total weight of the oily phase, of at least one enteric coating material, and
2-25 wt-%, based on the total weight of the oily phase, of at least one polyunsaturated fatty acid, and
optionally 1-25 wt-%, based on the total weight of the oily phase, of at least one saturated fatty acid, and
up to 15 wt-%, based on the total weight of the oily phase, of at least one further auxiliary agent.

Therefore, the present invention relates to a coating system (CS10′″), which is the coating system (CS), (CS1), (CS2), (CS2′), (CS3), (CS4), (CS5), (CS5′), (CS6), (CS6′), (CS6″), (CS7), (CS7′), (CS8), (CS9), (CS9′), (CS9″), (CS9′″), (CS9″ ″) or (CS9″″′), wherein the oily phase of the emulsion comprises
60-95 wt-%, based on the total weight of the oily phase, of at least one enteric coating material, and
2-20 wt-%, based on the total weight of the oily phase, of at least one polyunsaturated fatty acid, and
optionally 1-20 wt-%, based on the total weight of the oily phase, of at least one saturated fatty acid, and
up to 15 wt-%, based on the total weight of the oily phase, of at least one further auxiliary agent.

Therefore, the present invention relates to a coating system (CS10″ ″), which is the coating system (CS), (CS1), (CS2), (CS2′), (CS3), (CS4), (CS5), (CS5′), (CS6), (CS6′), (CS6″), (CS7), (CS7′), (CS8), (CS9), (CS9′), (CS9″), (CS9′″), (CS9″ ″) or (CS9″″′), wherein the oily phase of the emulsion comprises
60-95 wt-%, based on the total weight of the oily phase, of at least one enteric coating material, and
2-20 wt-%, based on the total weight of the oily phase, of at least one polyunsaturated fatty acid, and
optionally 1-20 wt-%, based on the total weight of the oily phase, of at least one saturated fatty acid.

Therefore, the present invention relates to a coating system (CS10″″′), which is the coating system (CS), (CS1), (CS2), (CS2′), (CS3), (CS4), (CS5), (CS5′), (CS6), (CS6′), (CS6″), (CS7), (CS7′), (CS8), (CS9), (CS9′), (CS9″), (CS9′″), (CS9″ ″) or (CS9″″′), wherein the oily phase of the emulsion consists of
60-95 wt-%, based on the total weight of the oily phase, of at least one enteric coating material, and
2-20 wt-%, based on the total weight of the oily phase, of at least one polyunsaturated fatty acid, and
1-20 wt-%, based on the total weight of the oily phase, of at least one saturated fatty acid, and
up to 15 wt-%, based on the total weight of the oily phase, of at least one further auxiliary agent.

Therefore, the present invention relates to a coating system (CS10″″″), which is the coating system (CS), (CS1), (CS2), (CS2′), (CS3), (CS4), (CS5), (CS5′), (CS6), (CS6′), (CS6″), (CS7), (CS7′), (CS8), (CS9), (CS9′), (CS9″), (CS9′″), (CS9″″) or (CS9″″′), wherein the oily phase of the emulsion consists of
60-95 wt-%, based on the total weight of the oily phase, of at least one enteric coating material, and
2-20 wt-%, based on the total weight of the oily phase, of at least one polyunsaturated fatty acid, and
optionally 1-20 wt-%, based on the total weight of the oily phase, of at least one saturated fatty acid.

All percentages are adding up always to 100% (this applies to all compositions disclosed in this patent application).

Therefore, the present invention relates to a coating system (CS11), which is the coating system (CS), (CS1), (CS2), (CS2′), (CS3), (CS4), (CS5), (CS5′), (CS6), (CS6′), (CS6″), (CS7), (CS7′), (CS8), (CS9), (CS9′), (CS9″), (CS9″ ″), (CS10), (CS10′), (CS10″), (CS10′″) or (CS10″″′), wherein the auxiliary agent is chosen from the group consisting of colors, antifoaming agents, flavors and antioxidants.

As stated above the particles are coated with the coating systems, which are the emulsions described above.

The emulsions used for the coating process should have a viscosity, which is suitable that the emulsion can be sprayed on the particles and that the spraying nozzle is not blocked.

A suitable range of viscosity goes up to 500 mPas. (measured at 45° C. (spraying temperature) using a Brookfield rheometer) Therefore, the present invention relates to a coating system (CS12), which is the coating system (CS), (CS1), (CS2), (CS2′), (CS3), (CS4), (CS5), (CS5′), (CS6), (CS6′), (CS6″), (CS7), (CS7′), (CS8), (CS9), (CS9′), (CS9″), (CS9′″), (CS9″ ″), (CS9″″′), (CS10), (CS10′), (CS10″), (CS10′″), (CS10″″′), (CS10″″′), (CS10″″ ″) or (CS11), wherein the emulsion has a viscosity of up to 500 mPas.

The coating process can be done by using commonly known processes and equipment. Also, the process conditions applied are commonly known and vary depending which process and/or equipment is used.

Typical coating techniques for coating of such tablets are i.e. dip coating, spray coating, film coating fluid bed coating, drum coating and pan coating

It is not essential for the present invention, which process is used.

Preferably, spray coating, drum coating or film coating is used.

Any suitable coating device can be used.

Such equipment is commercially available from a variety of suppliers, such i.e. Glatt (CH), Comasa etc.

The coating process is usually carried out as following
(i) the emulsion (CS), (CS1), (CS2), (CS2′), (CS3), (CS4), (CS5), (CS5′), (CS6), (CS6′), (CS6″), (CS7), (CS7′), (CS8), (CS9), (CS9′), (CS9″), (CS9′″), (CS9″ ″), (CS9″″′), (CS10), (CS10′), (CS10″), (CS10′″), (CS10″″′), (CS10″″′), (CS10″″ ″), (CS11) or (CS12) is prepared by preparing each of the phases, which are then emulsified, then
(ii) the emulsion is sprayed onto the particles (at a temperature range of 20° C. to 70° C.), then
(iii) the coated particles are collected and then
(iv) optionally the coated particles are undergoing a curing process.

As stated above, the process parameters chosen for the coating are commonly known and used for such a process.

The curing process is usually done by heating the particles up to 90° C. for a period of at least 1 minute. The maximal duration of the curing step is not essential, but it is usually not more than one hour.

Therefore, the curing step is carried for 1 minute to up to one hour.

Preferably the coated particles are undergoing a curing process step.

The coating process is usually carried out as following
(i) the emulsion (CS), (CS1), (CS2), (CS2′), (CS3), (CS4), (CS5), (CS5′), (CS6), (CS6′), (CS6″), (CS7), (CS7′), (CS8), (CS9), (CS9′), (CS9″), (CS9′″), (CS9″ ″), (CS9″″′), (CS10), (CS10′), (CS10″), (CS10′″), (CS10″″′), (CS10″″′), (CS10″″ ″), (CS11) or (CS12) is prepared by preparing each of the phases, which are then emulsified, then
(ii) the emulsion is sprayed onto the particles (at a temperature range of 20° C. to 70° C.), then
(iii) the coated particles are collected and then
(iv) the coated particles are undergoing a curing process at a temperature of up to 90° C. for at least one minute (preferably 1 minute to up to one hour).

When the coating system is applied onto the particle, the water of the emulsion is (more or less) evaporated. This means that the water content of the coating layer of coated particle is less than 5 wt-%, based on the total weight of the coated particles (preferably below 3 wt-%, more preferred below 2 wt-%, even more preferred below 1 wt-%).

The coated particles according to the present invention consists of a core (which is the particle) and the coating layer.

The coated particle according to the present invention consist of
(a) 70 to 99 wt-%, based on the total weight of the coated particle, of the particle comprising at least one active ingredient, and
(b) 1 to 30 wt-%, based on the total weight of the coated particle, of the coating layer, wherein the water content of the coated particle is less than 5 wt-%, based on the total weight of the coated particles (preferably below 3 wt-%, more preferred below 2 wt-%, even more preferred below 1 wt-%).

Therefore, the present invention relates to a coated particle (CP) consisting of
(a) 70 to 99 wt-%, based on the total weight of the coated particle, of the particle comprising at least one active ingredient, and
(b) 1 to 30 wt-%, based on the total weight of the coated particle, of the coating layer, wherein the water content of the coated particle is less than 5 wt-%, based on the total weight of the coated particles (preferably below 3 wt-%, more preferred below 2 wt-%, even more preferred below 1 wt-%).

As stated above the coating layer is formed by the coating system according to the present invention. Due to the process conditions of the coating process, the water of the coating system is removed (more or less) completely.

Therefore, the present invention relates to a coated particle (CP1) consisting of
(a) 70 to 99 wt-%, based on the total weight of the coated particle, of the particle comprising at least one active ingredient, and
(b) 1 to 30 wt-%, based on the total weight of the coated particle, of the coating layer, which comprises
   a. 1-8 wt-%, based on the total weight of the coating layer, of at least one emulsifier, and
   b. 1-8 wt-%, based on the total weight of the coating layer, of at least one plasticizer, and
   c. 2-20 wt-%, based on the total weight of the coating layer, of at least one film forming agent, and
   d. 2-20 wt-%, based on the total weight of the coating layer, of at least one pH adjuster, and
   e. 50-80 wt-%, based on the total weight of the coating layer, of at least one enteric coating material, and
   f. 2-15 wt-%, based on the total weight of the coating layer, of at least one polyunsaturated fatty acid, and
   g. optionally 1-15 wt-%, based on the total weight of the coating layer, of at least one saturated fatty, and
   h. up to 15 wt-%, based on the total weight of the coating layer, of at least one further auxiliary agent.

Therefore, the present invention relates to a coated particle (CP1′) consisting of
(a) 70 to 99 wt-%, based on the total weight of the coated particle, of the particle comprising at least one active ingredient, and
(b) 1 to 30 wt-%, based on the total weight of the coated particle, of the coating layer, which consists of
   a. 1-8 wt-%, based on the total weight of the coating layer, of at least one emulsifier, and
   b. 1-8 wt-%, based on the total weight of the coating layer, of at least one plasticizer, and
   c. 2-20 wt-%, based on the total weight of the coating layer, of at least one film forming agent, and
   d. 2-20 wt-%, based on the total weight of the coating layer, of at least one pH adjuster, and
   e. 50-80 wt-%, based on the total weight of the coating layer, of at least one enteric coating material, and
   f. 2-15 wt-%, based on the total weight of the coating layer, of at least one polyunsaturated fatty acid, and
   g. optionally 1-15 wt-%, based on the total weight of the coating layer, of at least one saturated fatty, and
   h. up to 15 wt-%, based on the total weight of the coating layer, of at least one further auxiliary agent.

Therefore, the present invention relates to a coated particle (CP1″) consisting of
(a) 70 to 99 wt-%, based on the total weight of the coated particle, of the particle comprising at least one active ingredient, and
(b) 1 to 30 wt-%, based on the total weight of the coated particle, of the coating layer, which consists of
   a. 1-8 wt-%, based on the total weight of the coating layer, of at least one emulsifier, and
   b. 1-8 wt-%, based on the total weight of the coating layer, of at least one plasticizer, and
   c. 2-20 wt-%, based on the total weight of the coating layer, of at least one film forming agent, and
   d. 2-20 wt-%, based on the total weight of the coating layer, of at least one pH adjuster, and
   e. 50-80 wt-%, based on the total weight of the coating layer, of at least one enteric coating material, and
   f. 2-15 wt-%, based on the total weight of the coating layer, of at least one polyunsaturated fatty acid, and
   g. optionally 1-15 wt-%, based on the total weight of the coating layer, of at least one saturated fatty.

Preferably, the present invention relates to a coated particle (CP1′″) consisting of
(a) 70 to 99 wt-%, based on the total weight of the coated particle, of the particle comprising at least one active ingredient, and
(b) 1 to 30 wt-%, based on the total weight of the coated particle, of the coating layer, which comprises
   a. 1-5 wt-%, based on the total weight of the coating layer, of at least one emulsifier, and
   b. 1-5 wt-%, based on the total weight of the coating layer, of at least one plasticizer, and
   c. 2-20 wt-%, based on the total weight of the coating layer, of at least one film forming agent, and
   d. 4-15 wt-%, based on the total weight of the coating layer, of at least one pH adjuster, and
   e. 50-75 wt-%, based on the total weight of the coating layer, of at least one enteric coating material, and
   f. 2-10 wt-%, based on the total weight of the coating layer, of at least one polyunsaturated fatty acid, and
   g. optionally 1-10 wt-%, based on the total weight of the coating layer, of at least one saturated fatty, and
   h. up to 15 wt-%, based on the total weight of the coating layer, of at least one further auxiliary agent.

Preferably, the present invention relates to a coated particle (CP1″″′) consisting of
(a) 70 to 99 wt-%, based on the total weight of the coated particle, of the particle comprising at least one active ingredient, and
(b) 1 to 30 wt-%, based on the total weight of the coated particle, of the coating layer, which consists of
   a. 1-5 wt-%, based on the total weight of the coating layer, of at least one emulsifier, and
   b. 1-5 wt-%, based on the total weight of the coating layer, of at least one plasticizer, and
   c. 2-20 wt-%, based on the total weight of the coating layer, of at least one film forming agent, and
   d. 4-15 wt-%, based on the total weight of the coating layer, of at least one pH adjuster, and
   e. 50-75 wt-%, based on the total weight of the coating layer, of at least one enteric coating material, and
   f. 2-10 wt-%, based on the total weight of the coating layer, of at least one polyunsaturated fatty acid, and
   g. optionally 1-10 wt-%, based on the total weight of the coating layer, of at least one saturated fatty, and h. up to 15 wt-%, based on the total weight of the coating layer, of at least one further auxiliary agent.

Preferably, therefore, the present invention relates to a coated particle (CP1″″ ″) consisting of
(a) 70 to 99 wt-%, based on the total weight of the coated particle, of the particle comprising at least one active ingredient, and
(b) 1 to 30 wt-%, based on the total weight of the coated particle, of the coating layer, which consists of
   a. 1-5 wt-%, based on the total weight of the coating layer, of at least one emulsifier, and
   b. 1-5 wt-%, based on the total weight of the coating layer, of at least one plasticizer, and
   c. 2-20 wt-%, based on the total weight of the coating layer, of at least one film forming agent, and
   d. 4-15 wt-%, based on the total weight of the coating layer, of at least one pH adjuster, and
   e. 50-75 wt-%, based on the total weight of the coating layer, of at least one enteric coating material, and
   f. 2-10 wt-%, based on the total weight of the coating layer, of at least one polyunsaturated fatty acid, and
   g. optionally 1-10 wt-%, based on the total weight of the coating layer, of at least one saturated fatty.

Therefore, the present invention relates to a coated particle (CP2), which is the coated particle (CP1), (CP1′), (CP1′″) or (CP1″″), wherein the auxiliary agent is chosen from the group consisting of colors, antifoaming agents, flavors and antioxidant.

Therefore, the present invention relates to a coated particle (CP3), which is the coated particle (CP), (CP1), (CP1′), (CP1″), (CP1′″), (CP1″ ″), (CP1″″′), (CP1″″ ″) or (CP2), wherein the at least one emulsifier is gelatine.

The gelatine can be from any source, such as porcine (pork), bovine (beef or cattle), and fish.

Therefore, the present invention relates to a coated particle (CP4), which is coated particle (CP), (CP1), (CP1′), (CP1″), (CP1′″), (CP1″ ″), (CP1″″′), (CP1″″ ″), (CP2) or (CP3), wherein the at least one plasticizer is chosen from the group consisting of diesters of acids, triesters of acids, diesters of alcohols, triesters of alcohols, polyols and polyethylene glycols.

Therefore, the present invention relates to a coated particle (CP4′), which is the coated particle (CP), (CP1), (CP1′), (CP1″), (CP1′″), (CP1″ ″), (CP1″″′), (CP1″″ ″), (CP2) or (CP3), wherein the at least one plasticizer is chosen from the group consisting of triethyl citrate, tributyl citrate, acetyl triethyl citrate and glycerol.

Therefore, the present invention relates to a coated particle (CP5), which is the coated particle (CP), (CP1), (CP1′), (CP1″), (CP1′″), (CP1″ ″), (CP1″″′), (CP1″″ ″), (CP2), (CP3), (CP4) or (CP4′), wherein the at least one film forming agent is shellac and/or shellac ammonium salt, polyvinylpyrrolidone (PVP), acrylates, acrylamides, and copolymers thereof

Therefore, the present invention relates to a coated particle (CP6), which is the coated particle (CP), (CP1), (CP1′), (CP1″), (CP1′″), (CP1″ ″), (CP1″″′), (CP1″″ ″), (CP2), (CP3), (CP4), (CP4′), or (CP5), wherein the at least one pH adjuster is chosen from the group consisting of NaOH and KOH.

Therefore, the present invention relates to a coated particle (CP7), which is the coated particle (CP), (CP1), (CP1′), (CP1″), (CP1′″), (CP1″ ″), (CP1″″′), (CP1″″ ″), (CP2), (CP3), (CP4), (CP4′), (CP5) or (CP6), wherein the at least one enteric coating is a wax.

Therefore, the present invention relates to a coated particle (CP7′), which is the coated particle (CP), (CP1), (CP1′), (CP1″), (CP1′″), (CP1″ ″), (CP1″″′), (CP1″″ ″), (CP2), (CP3), (CP4), (CP4′), (CP5) or (CP6), wherein the enteric coating is Candelilla wax.

Candelilla wax is a wax derived from the leaves of the small Candelilla shrub native to northern Mexico and the southwestern United States, Euphorbia Cerifera and Euphorbia antisyphilitica, from the family Euphorbiaceae. It is yellowish-brown, hard, brittle, aromatic, and opaque to translucent. Candelilla wax consists of mainly hydrocarbons (about 50%, chains with 29-33 carbons), esters of higher molecular weight (20-29%), free acids (7-9%), and resins (12-14%, mainly triterpenoid esters) Therefore, the present invention relates to a coated particle (CP8), which is the coated particle (CP), (CP1), (CP1′), (CP1″), (CP1′″), (CP1″ ″), (CP1″″′), (CP1″″ ″), (CP2), (CP3), (CP4), (CP4′), (CP5), (CP6), (CP7) or (CP7′), wherein the at least one polyunsaturated fatty acid has 16 to 24 carbon atoms.

Therefore, the present invention relates to a coated particle (CP8′), which is the coated particle (CP), (CP1), (CP1′), (CP1″), (CP1′″), (CP1″ ″), (CP1″″′), (CP1″″ ″), (CP2), (CP3), (CP4), (CP4′), (CP5), (CP6), (CP7) or (CP7′), wherein the at least one polyunsaturated fatty acid is chosen from the group consisting of omega-3 fatty acids, omega-6 fatty acids, omega-9 fatty acids and conjugated fatty acids.

Therefore, the present invention relates to a coated particle (CP8″), which is the coated particle (CP), (CP1), (CP1′), (CP1″), (CP1′″), (CP1″ ″), (CP1″″′), (CP1″″ ″), (CP2), (CP3), (CP4), (CP4′), (CP5), (CP6), (CP7) or (CP7′), wherein the at least one polyunsaturated fatty acid is chosen from the group consisting of oleic acid and linoleic acid.

Therefore, the present invention relates to a coated particle (CP9), which is the coated particle (CP), (CP1), (CP1′), (CP1″), (CP1′″), (CP1″ ″), (CP1″″′), (CP1″″ ″), (CP2), (CP3), (CP4), (CP4′), (CP5), (CP6), (CP7), (CP7′), (CP8), (CP8′) or (CP8″), wherein the at least one saturated fatty acid has 16 to 24 carbon atoms.

Therefore, the present invention relates to a coated particle (CP9′), which is the coated particle (CP), (CP1), (CP1′), (CP1″), (CP1′″), (CP1″ ″), (CP1″″′), (CP1″″ ″), (CP2), (CP3), (CP4), (CP4′), (CP5), (CP6), (CP7), (CP7′), (CP8), (CP8′) or (CP8″), wherein the at least one saturated fatty acid is chosen from the group consisting of lauric acid, myristic acid, palmitic acid and stearic acid.

The particles, which are coated by the inventive coating, may have any size and shape, which are meant to be consumed.

As stated above, the shape of the particle (which is the core of the coated particle) does not change substantially when it is coated.

The particles may have a size, which is commonly known for the type of particles. They may be in the form of a monolithic dosage form or multiparticulates (or multiple unit dosage form).

Therefore, the present invention relates to coating particles (CP10), which are the coated particles (CP), (CP1), (CP1′), (CP1″), (CP1′″), (CP1″ ″), (CP1″″′), (CP1″″ ″), (CP2), (CP3), (CP4), (CP4′), (CP5), (CP6), (CP7), (CP7′), (CP8), (CP8′), (CP8″), (CP9) or (CP9′), wherein the coated particles are a monolithic dosage form or multiparticulates (or multiple unit dosage form).

The size of the largest dimension relates on the shape of the particle.

The shape can be any known and used, such as spheres, disc-like, bean-like, egg shape etc.

Therefore, a suitable size range goes from below 1 mm to about 1.5 cm (largest dimension).

Therefore, the present invention relates to a coating particle (CP11), which is the coated particle (CP), (CP1), (CP1′), (CP1″), (CP1′″), (CP1″ ″), (CP1″″′), (CP1″″ ″), (CP2), (CP3), (CP4), (CP4′), (CP5), (CP6), (CP7), (CP7′), (CP8), (CP8′), (CP8″), (CP9), (CP9′) or (CP10), wherein the coated particle has a size from below 1 mm to about 1.5 cm (largest dimension).

The particle, which is coated, can be a tablet, capsule, caplet, extrudate, pellet, globuli, granulate, beadlet, etc which comprises at least one active ingredient and optionally at least one further excipient, which are used to produce such a dosage form.

Therefore, the present invention relates to a coating particle (CP12), which is the coated particle (CP), (CP1), (CP1′), (CP1″), (CP1′″), (CP1″ ″), (CP1″″′), (CP1″″ ″), (CP2), (CP3), (CP4), (CP4′), (CP5), (CP6), (CP7), (CP7′), (CP8), (CP8′), (CP8″), (CP9), (CP9′), (CP10) or (CP11), wherein the particle, which is coated, is a tablet, capsule, caplet, extrudate, pellet, globuli, granulate, or a beadlet, which comprises at least one active ingredient and optionally at least one further excipient, which are used to produce such a dosage form.

The particle can also be a pure substance. This means no excipient is used.

The coating layer covers the particle (more or less) equally. This means that the particle is coated completely, and the thickness is more or less the same all over the particle.

The thickness of the coating layer goes from 20 m to 500 m (preferably 50 m to 500 m, more preferably 100 m to 400 m).

The thickness is measured by commonly known methods, such as i.e., scanning electron microscopy.

Therefore, the present invention relates to a coating particle (CP13), which is the coated particle (CP), (CP1), (CP1′), (CP1″), (CP1′″), (CP1″ ″), (CP1″″′), (CP1″″ ″), (CP2), (CP3), (CP4), (CP4′), (CP5), (CP6), (CP7), (CP7′), (CP8), (CP8′), (CP8″), (CP9), (CP9′), (CP10), (CP11) or (CP12), wherein the thickness of the coating is 20 m to 500 μm (preferably 50 m to 500 m, more preferably 100 μm to 400 m).

The active ingredient in the particle can be any suitable one depending on the desired use of the coated particle.

The active ingredient can be a i.e., pharmaceutical compound (a drug), a vitamin, a mineral, probiotic, nutritional lipid, enzyme, eubiotic, plant extract and nutraceutical.

Therefore, the present invention relates to a coating particle (CP14), which is the coated particle (CP), (CP1), (CP1′), (CP1″), (CP1′″), (CP1″ ″), (CP1″″′), (CP1″″ ″), (CP2), (CP3), (CP4), (CP4′), (CP5), (CP6), (CP7), (CP7′), (CP8), (CP8′), (CP8″), (CP9), (CP9′), (CP10), (CP11), (CP12) or (CP13), wherein the active ingredient in the particle is chosen from the group consisting of pharmaceutical compound (a drug), a vitamin, a mineral, probiotic, nutritional lipid, enzyme, eubiotic, plant extract and nutraceutical.

When using a vitamin, it can be water and/or fat-soluble vitamin.

Water soluble vitamins are ascorbic acid (vitamin C), thiamin, riboflavin, niacin, vitamin B6 (pyridoxine, pyridoxal, and pyridoxamine), folacin, vitamin B12, biotin, and pantothenic acid.

Fat soluble vitamins are A, vitamin D, vitamin E and vitamin K.

The amount of the at least one active ingredient in the particle can vary depending on the active ingredient and/or on the use of the active ingredient and/or on the dosage regime of the final formulation (which is consumed), etc.

Depending on the active ingredient in the particle (which is the core of the coated particle), the coated particle can be used in various fields of application, such as in a dietary supplement formulation, in a pharmaceutical formulation, in a personal care formulation, in food formulation and feed formulation.

Therefore, the present invention relates to the use of at least one coated particle, which is the coated (CP), (CP1), (CP1′), (CP1″), (CP1′″), (CP1″ ″), (CP1″″′), (CP1″″ ″), (CP2), (CP3), (CP4), (CP4′), (CP5), (CP6), (CP7), (CP7′), (CP8), (CP8′), (CP8″), (CP9), (CP9′), (CP10), (CP11), (CP12), (CP13) or (CP14), in a dietary supplement formulation, in a pharmaceutical formulation, in a personal care formulation, in food formulation and/or feed formulation.

The coated particle according to the present invention can be used as such, or it can be incorporated into a formulation, which can be any commonly known formulation (preferably, it is a dry formulation).

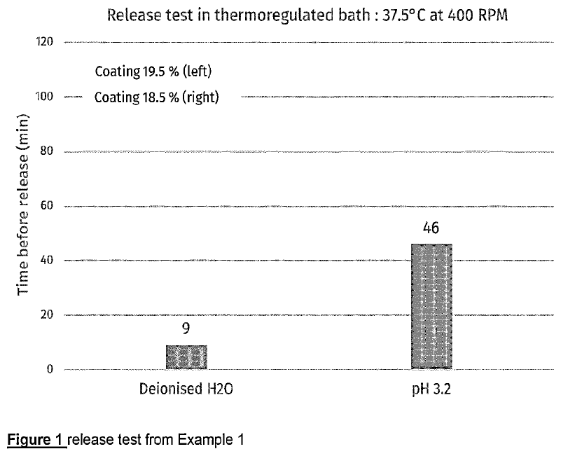

FIG. 1: release test from Example 1

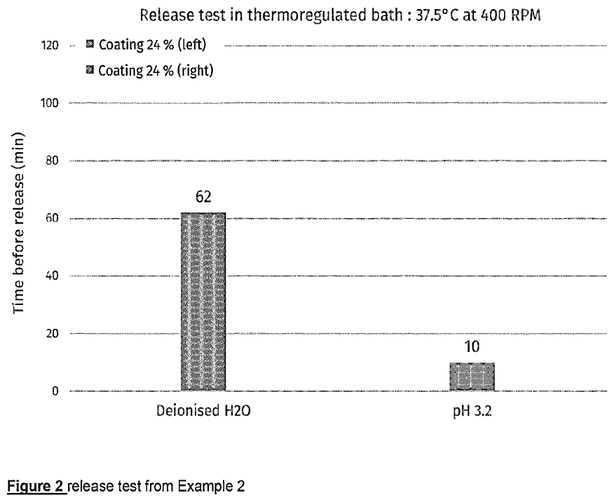

FIG. 2: release test from Example 2

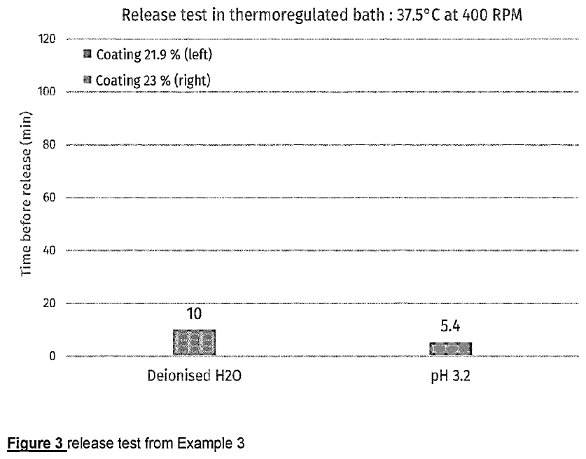

FIG. 3: release test from Example 3

The following examples serve to illustrate specific embodiments of the invention claimed herein. All percentages are given in relation to the weight, and all the temperatures are given in degrees Celsius.

### EXAMPLES

### Example 1

### Aqueous Phase Preparation:

25 g H₂O and 4.79 g gelatine and 25 g H₂O were firstly introduced in a 800 ml glass beaker. Then, the mixture was left 1 h in a water bath at 35° C.

After 1 h, the gelatine mixture was immersed in a water bath at 68° C. before to add the remaining amount of H₂O (150 g) and the glycerol (4.79 g). The mixture was mixed and it was stirred at low speed.

Then, the shellac ammonium salt solution (73.32 g) was added.

Once homogeneous, the pH of the solution was measured and adjusted to 7.89 by addition of 20.77 g of NaOH 30% sol. The solution was placed again the hot bath under stirring.

### Oily Phase Preparation:

13.5 g of oleic acid and 6.75 g of stearic acid were firstly introduced in a 250 mL glass beaker. After addition of 135 g of melted wax, the mixture was quickly placed in a water bath at 90° C. under magnetic stirring to mix the different products.

### Emulsion Preparation:

Once the temperature of the aqueous phase reached 65° C., the homogenizer speed (UItraturrax T25) was progressively increased until 24000 rpm while adding the oily phase slowly.

The emulsion was finally sieved using a sieve (mesh size 500 μm).

### Coating:

The coating was performed in a drumcoater Glatt GC 1 equipped with a 0.8 L drum and with the standard 0.8 mm nozzle. A Silicone tubing (2×2×6) from VWR was used to ensure the transportation of the emulsion from the bottle to the nozzle. A water bath allowed to keep the emulsion hot and under stirring during the coating. The drum was filled with 300 g of vitamin B2 Tablets.

The following table shows the composition of the coating system used in Example 1

| Phase | Ingredients | Amount (% w/w) |
|---|---|---|
| Aqueous | Water | 58.7 |
| | Gelatin | 1 |
| | Glycerol | 1 |
| | Shellac 25% solution | 4 |
| | NaOH | 1.4 |
| Oily | Candelilla wax | 29.4 |
| | Oleic acid | 2.9 |
| | Stearic acid | 1.5 |

Release tests realized in a thermoregulated bath (37.5° C.) under stirring (400 RPM) in two different mediums, namely in deionised water and in an acidic buffer solution at pH 3.2

### Example 2: Composition without Shellac (Comparison Example)

This example was made in accordance to Example 1

The following table shows the composition of the coating system used in Example 2

| Phase | Ingredients | Amount (% w/w) |
|---|---|---|
| Aqueous | Water | 56.4 |
| | Gelatin | 9.4 |
| | Glycerol | 1 |
| | NaOH | 1.9 |
| Oily | Candelilla wax | 28.1 |
| | Oleic acid | 3.1 |

Release tests realized in a thermoregulated bath (37.5° C.) under stirring (400 RPM) in two different mediums (deionised water and acidic buffer solution):

### Example 3: Composition with HPMC (Hydroxypropylmethylcellulose) Instead of Gelatin (Comparison Example)

This example was made in accordance to Example 1

The following table shows the composition of the coating system used in Example 3

| Phase | Ingredients | Amount (% w/w) |
|---|---|---|
| Aqueous | Water | 61 |
| | HPMC | 6.8 |
| | Glycerol | 0.9 |
| | NaOH | Few droplets |
| Oily | Candelilla wax | 28.2 |
| | Oleic acid | 3 |

Release tests realized in a thermoregulated bath (37.5° C.) under stirring (400 RPM) in two different mediums (deionised water and acidic buffer solution):

It can be seen that the examples of the present invention show a much better release properties than the Comparison Example.

1. A coating system consisting of an
(i) aqueous solution comprising
a. water and
b. at least one emulsifier, and
c. at least one plasticizer, and
d. at least one film forming agent, and
e. at least one pH adjuster, and
(ii) oily phase comprising
a. at least one enteric coating material, and
b. at least one polyunsaturated fatty acid, and
c. optionally at least one saturated fatty acid.

2. Coating system according to claim 1, wherein the at least one emulsifier is gelatine.

3. Coating system according to claim 1, wherein the at least one plasticizer is chosen from the group consisting of diesters of acids, triesters of acids, diesters of alcohols, triesters of alcohols, polyols and polyethylene glycols.

4. Coating system according to claim 1, wherein the at least one film forming agent is shellac and/or shellac ammonium salt, polyvinylpyrrolidone (PVP), acrylates, acrylamides, and copolymers thereof.

5. Coating system according to claim 1, wherein the at least one pH adjuster is chosen from the group consisting of NaOH and KOH.

6. Coating system according to claim 1, wherein the at least one enteric coating is a wax.

7. Coating system according to claim 6, wherein the enteric coating is Candelilla wax.

8. Coating system according to claim 1, wherein the at least one polyunsaturated fatty acid has 16 to 24 carbon atoms.

9. Coating system according to claim 1, wherein the at least one saturated fatty acid has 16 to 24 carbon atoms.

10. Coating system according to claim 1, wherein the emulsion consists of 50-80 wt-%, based on the total weight of the emulsion, of the aqueous (water) phase and 20-50 wt-%, based on the total weight of the emulsion, of the oily phase.

11. Coating system according to claim 1, wherein the aqueous phase of the emulsion comprises
65-90 wt-%, based on the total weight of the aqueous phase, of water and
0.5-10 wt-%, based on the total weight of the aqueous phase, of at least one emulsifier, and
0.5-10 wt-%, based on the total weight of the aqueous phase, of at least one plasticizer, and
2-15 wt-%, based on the total weight of the aqueous phase, of at least one film forming agent, and
2-15 wt-%, based on the total weight of the aqueous phase, of at least one pH adjuster, and
up to 15 wt-%, based on the total weight of the aqueous phase, of at least one further auxiliary agent.

12. Coating system according to claim 1,
wherein the aqueous phase of the emulsion consists of
60-95 wt-%, based on the total weight of the oily phase, of at least one enteric coating material, and
2-20 wt-%, based on the total weight of the oily phase, of at least one polyunsaturated fatty acid, and
optionally 1-20 wt-%, based on the total weight of the oily phase, of at least one saturated fatty acid, and
up to 15 wt-%, based on the total weight of the oily phase, of at least one further auxiliary agent.

13. Coating process wherein
(i) an emulsion of claim 1 is prepared by preparing each of the phases, which are then emulsified, then
(ii) the emulsion is sprayed onto the particles (at a temperature range of 20° C. to 50° C.), then
(iii) the particles are collected and then
(iv) optionally the particles are undergoing a curing process.

14. A coated particle consisting of
(a) 70 to 99 wt-%, based on the total weight of the coated particle, of the particle comprising at least one active ingredient, and
(b) 1 to 30 wt-%, based on the total weight of the coated particle, of the coating layer, which comprises
a. 1-8 wt-%, based on the total weight of the coating layer, of at least one emulsifier, and
b. 1-8 wt-%, based on the total weight of the coating layer, of at least one plasticizer, and
c. 2-20 wt-%, based on the total weight of the coating layer, of at least one film forming agent, and
d. 2-20 wt-%, based on the total weight of the coating layer, of at least one pH adjuster, and
e. 50-80 wt-%, based on the total weight of the coating layer, of at least one enteric coating material, and
f. 2-15 wt-%, based on the total weight of the coating layer, of at least one polyunsaturated fatty acid, and
g. optionally 1-15 wt-%, based on the total weight of the coating layer, of at least one saturated fatty, and
h. up to 15 wt-%, based on the total weight of the coating layer, of at least one further auxiliary agent.
